(19) United States
(12) Patent Application Publication
(10) Pub. No.: US 2019/0099424 A1
(43) Pub. Date: Apr. 4, 2019

(54) Pharmaceutical Composition for Decreasing The Side Effects of Pancreatic Cancer Drug, and Manufacturing Method and Uses Thereof

(71) Applicant: Dai-Ming Kuo, New Taipei City (TW)

(72) Inventor: Dai-Ming Kuo, New Taipei City (TW)

(21) Appl. No.: 16/207,767

(22) Filed: Dec. 3, 2018

Related U.S. Application Data
(63) Continuation-in-part of application No. 15/285,846, filed on Oct. 5, 2016.

(30) Foreign Application Priority Data
Jun. 15, 2016 (TW) 105118779
Dec. 14, 2017 (TW) 106143997

(51) Int. Cl. A61K 31/519; A61K 36/03; A61K 36/068; A61K 36/074; A61K 36/481; A61K 36/537; A61K 36/344; A61K 36/748; A61K 36/46; A61K 36/284; A61K 36/254; A61K 36/41; A61K 36/8998; A61K 36/8968; A61K 36/79; A61K 36/899; A61K 36/48; A61K 36/87; A61K 36/21; A61K 36/31; A61K 36/07; A61K 36/062; A61K 36/42; A61K 36/287; A61K 36/355; A61P 35/00; A61K 31/44; A61K 31/53; A61K 31/4745; A61K 31/513

(52) U.S. Cl. CPC A61K 31/519; A61K 31/513; A61K 36/068; A61K 36/074; A61K 36/481; A61K 36/537; A61K 36/344; A61K 36/748; A61K 36/46; A61K 36/284; A61K 36/254; A61K 36/41; A61K 36/8998; A61K 36/8968; A61K 36/79; A61K 36/899; A61K 36/48; A61K 36/87; A61K 36/21; A61K 36/31; A61K 36/07; A61K 36/062; A61K 36/42; A61K 36/287; A61K 36/355; A61P 35/00; A61K 31/44; A61K 31/53; A61K 31/4745; A61K 36/03

(57) ABSTRACT

The present invention provides a pharmaceutical composition comprising a mushroom, a rhizome, a fruit, a leaf, a flower, an alga, an energy-rich liquid, a salt-rich liquid, an assist agent and an anti-oxide agent. Said pharmaceutical composition has ability to improve autoimmunity, protect from the side-effects caused by chemical cancer drugs and increase the functions of chemical pancreatic cancer drugs.

### CROSS-REFERENCE TO RELATED APPLICATIONS

This application is a Continuation-in-Part of co-pending application Ser. No. 15/285,846, filed on Oct. 5, 2016, for which priority is claimed under 35 U.S.C. § 120; and this application claims priority of Application No. 105118779 filed in Taiwan on Jun. 15, 2016 and Application No. 106143997 filed in Taiwan on Dec. 14, 2017 under 35 U.S.C. § 119; the entire contents of all of which are hereby incorporated by reference.

### FIELD OF THE INVENTION

The present invention relates to a pharmaceutical composition for treating pancreatic cancer, especially related to a pharmaceutical composition can combine with chemotherapeutic drugs for treating pancreatic cancer.

### BACKGROUND OF THE INVENTION

Chemotherapy is an important clinical treatment for cancer patients, which is through administering anticancer drugs to patients to kill cancer cells. In comparison with the conventional surgery and radiation therapy, the advantage of chemotherapy is to achieve systemic treatment. Therefore, chemotherapy becomes the major cancer treatment currently, and is the most important treatment for metastatic cancer patients.

The marketed chemotherapeutic drugs can be divided into chemical synthesized drugs and targeted drugs. The chemical synthesized drugs, including Bleomycin, Cisplatin, Fluorouracil (5-FU), Methotrexate, Taxol, are used to attack the cells which divided rapidly. Hence, in addition to cancer cells, the normal cells, such as white blood cells, red blood cells, platelets, hair follicle cells, gastrointestinal epithelial cells, germ cells, are also the object of chemical synthesized drugs. Consequently, chemical synthesized drugs usually causes side effects including nausea, vomiting, bleeding, diarrhea, hair loss, reproductive ability reduce, mucosal damage and inflamed, and make patients become easily infected.

Targeted drugs, including Bevacizumab, Iressa and Erlotinib, are acting on specific antigens or proteins of the cancer cells. Therefore, the targeted drugs are only able to treat the primary site of cancer cells, but not able to treat the metastatic cancer cells. Besides, targeted drugs also cause some side effects such as skin rash, pain, stomatitis, diarrhea, fever, high blood pressure, liver damage and other side effects.

In addition, some chemotherapeutic drugs, such as Fluorouracil (5-FU), Taxol, can even cause patients with arrhythmia, liver and kidney dysfunction, pain, hypertrophy etc., thus seriously affecting the life quality of cancer patients; TS-1 It may cause leukopenia, neutropenia, anemia, thrombocytopenia, elevated AST/ALT, loss of appetite, nausea, vomiting, diarrhea, generalized burnout, endophthalmitis, pigmentation, rashes, reactivation of hepatitis B Side effects.

In recent days, various countries are still devoted to studying the treatment of cancer drugs, and further experiments and development. However, while research and development of new drugs, the side effects of the drugs are still problems of current chemotherapy drugs, and further research and development are needed.

### SUMMARY OF THE INVENTION

Accordingly, the present invention provides a pharmaceutical composition could combine with chemotherapy drugs for treating pancreatic cancer and has ability to decrease the side effects of pancreatic cancer drug. Said pharmaceutical composition comprising a mushroom, a rhizome, a fruit, a leaf, a flower, an alga, an energy-rich liquid, a salt-rich liquid, an assist agent and an anti-oxide agent and the pancreatic cancer drug combination is selected from the group consisting of irinotecan (CPT 11), GEM, Leucovorin (LV), TS-1,5-FU and combinations thereof;

wherein the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof; wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof; wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof; wherein the leaves are selected from the group consisting of 8-12 grams of spinach, 8-12 grams of sprouted broccoli, 8-12 grams of papaya leaves, 6.4-9.6 g of Lotus leaf and combinations thereof; wherein the flower system is 8-12 g of Gongju chrysanthemum, 8-12 g of Huang Ju, 8-12 g of honeysuckle, 8-12 of chamomile G, or a combination thereof; wherein the algae is a group consisting of 8-12 grams of seaweed, 8-12 grams of konbu and 8-12 grams of kelp, or a combination thereof; wherein the energy configuration fluid comprises chlorine 4.5-5.5 mg of iron and 200 ml of distilled water; wherein, the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride; wherein the supplement 18-22 grams of citric acid, 18-22 grams of selenium yeast, 270-330 milligrams of coenzyme, and 2.7-3.3 grams of vitamin C; and the antioxidant comprises 9-11 grams of chitosan oligosaccharides, −5.5 grams, 0.9-1.1 grams of pine bark and 0.9-1.1 grams of brown algae polysaccharide.

In one of the embodiment, the pharmaceutical composition has a pH between 1.2-2.

In one of the embodiment, the pharmaceutical composition for pancreatic cancer further comprising leucovorin.

In one of the embodiment, the topoisomerase I inhibitor is irinotecan, SN-38 or topotecan, and the topoisomerase II inhibitor Is anthracycline, teniposide or etoposide and the antimetabolite is 5-Fluorouracil, capecitabine, Cytarabine, gemcitabine, methotrexate, pemetrexed or tegafur.

The present invention also provide a pharmaceutical composition for treating pancreatic cancer or decreasing the side effects of pancreatic cancer drug, wherein the pharmaceutical composition comprises a mushroom, a rhizome, a fruit, a leaf, a flower, algae, an energy-rich liquid, a salt-rich liquid, an assist agent and an anti-oxide agent;

In one of the embodiment, the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof; wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof; wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof; wherein the leaves are selected from the group consisting of 8-12 grams of spinach, 8-12 grams of sprouted broccoli, 8-12 grams of papaya leaves, 6.4-9.6 g of Lotus leaf and combinations thereof; wherein the flower system is 8-12 g of Gongju chrysanthemum, 8-12 g of Huang Ju, 8-12 g of honeysuckle, 8-12 of chamomile G, or a combination thereof; wherein the algae is a group consisting of 8-12 grams of seaweed, 8-12 grams of konbu and 8-12 grams of kelp, or a combination thereof; wherein the energy configuration fluid comprises chlorine 4.5-5.5 mg of iron and 200 ml of distilled water; wherein, the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride; wherein the supplement 18-22 grams of citric acid, 18-22 grams of selenium yeast, 270-330 milligrams of coenzyme, and 2.7-3.3 grams of vitamin C; and the antioxidant comprises 9-11 grams of chitosan oligosaccharides, −5.5 grams, 0.9-1.1 grams of pine bark and 0.9-1.1 grams of brown algae polysaccharide.

The application of claim 5, wherein the administration of the pharmaceutical compositions divided into 3 to 10 times a day and each time must be diluted 1/1000 to 3/1000 times.

In one of the embodiment, the pharmaceutical composition is used with a course of pancreatic cancer drugs.

In one of the embodiment, the administration of the pharmaceutical composition is administered daily for several days prior to the course of pancreatic cancer drugs, then treatment of the pancreatic cancer drugs begins on the day when the pharmaceutical composition is discontinued, and then continue to daily administration of the pharmaceutical composition after the pancreatic cancer drugs.

In one of the embodiment, the administration of the pharmaceutical composition is administered daily for several days prior to the course of pancreatic cancer drugs, then treatment of the pancreatic cancer drugs with the pharmaceutical composition, and then continue to daily administration of the pharmaceutical composition after the pancreatic cancer drugs.

In one of the embodiment, the administration of the pharmaceutical composition is administered daily for several days prior to the course of pancreatic cancer drugs, then treatment of the pancreatic cancer drugs with the pharmaceutical composition, and then continue to daily administration of the pharmaceutical composition after the pancreatic cancer drugs, sequential administration as above.

In one of the embodiment, the pharmaceutical composition for pancreatic cancer further comprising leucovorin.

In one of the embodiment, the topoisomerase I inhibitor is irinotecan, SN-38 or topotecan, and the topoisomerase II inhibitor Is anthracycline, teniposide or etoposide and the antimetabolite is 5-Fluorouracil, capecitabine, Cytarabine, gemcitabine, methotrexate, pemetrexed or tegafur.

The present invention also provides a pharmaceutical composition for treating pancreatic cancer or decreasing the side effects of pancreatic cancer drug, wherein the pharmaceutical composition comprises a mushroom, a rhizome, a fruit, a leaf, a flower, algae, an energy-rich liquid, a salt-rich liquid, an assist agent and an anti-oxide agent;

wherein the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof; wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof; wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof; wherein the leaves are selected from the group consisting of 8-12 grams of spinach, 8-12 grams of sprouted broccoli, 8-12 grams of papaya leaves, 6.4-9.6 g of Lotus leaf and combinations thereof; wherein the flower system is 8-12 g of Gongju chrysanthemum, 8-12 g of Huang Ju, 8-12 g of honeysuckle, 8-12 of chamomile G, or a combination thereof; wherein the algae is a group consisting of 8-12 grams of seaweed, 8-12 grams of konbu and 8-12 grams of kelp, or a combination thereof; wherein the energy configuration fluid comprises chlorine 4.5-5.5 mg of iron and 200 ml of distilled water; wherein, the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride; wherein the supplement 18-22 grams of citric acid, 18-22 grams of selenium yeast, 270-330 milligrams of coenzyme, and 2.7-3.3 grams of vitamin C; and the antioxidant comprises 9-11 grams of chitosan oligosaccharides, −5.5 grams, 0.9-1.1 grams of pine bark and 0.9-1.1 grams of brown algae polysaccharide.

In one of the embodiment, the administration of the pharmaceutical compositions divided into 3 to 10 times a day and each time must be diluted 1/1000 to 3/1000 times.

In one of the embodiment, the pharmaceutical composition is used with a course of pancreatic cancer drugs.

In one of the embodiment, the administration of the pharmaceutical composition is administered daily for several days prior to the course of pancreatic cancer drugs, then treatment of the pancreatic cancer drugs begins on the day when the pharmaceutical composition is discontinued, and then continue to daily administration of the pharmaceutical composition after the pancreatic cancer drugs.

In one of the embodiment, the administration of the pharmaceutical composition is administered daily for several days prior to the course of pancreatic cancer drugs, then treatment of the pancreatic cancer drugs with the pharmaceutical composition, and then continue to daily administration of the pharmaceutical composition after the pancreatic cancer drugs.

In one of the embodiment, the administration of the pharmaceutical composition is administered daily for several days prior to the course of pancreatic cancer drugs, then treatment of the pancreatic cancer drugs with the pharmaceutical composition, and then continue to daily administration of the pharmaceutical composition after the pancreatic cancer drugs, sequential administration as above.

In one of the embodiment, the pharmaceutical composition for pancreatic cancer further comprising leucovorin.

In one of the embodiment, the topoisomerase I inhibitor is irinotecan, SN-38 or topotecan, and the topoisomerase II inhibitor Is anthracycline, teniposide or etoposide and the antimetabolite is 5-Fluorouracil, capecitabine, Cytarabine, gemcitabine, methotrexate, pemetrexed or tegafur.

The details of one or more embodiments are set forth in the accompanying drawing and the description below. Other features, objects, and advantages of the embodiments will be apparent from the description and drawing, and from the claims.

### BRIEF DESCRIPTION OF THE DRAWINGS

FIG. 1 is a set of diagram that shows the drug treatment schedule of the 1st group to the 4th group in part I examples.

FIG. 2 is a set of diagram that shows the drug treatment schedule of the 5th group to the 7th in part I examples.

FIG. 3A-3B are bar graphs that illustrate the tumor volume of each group of mice in examples of the present invention.

FIG. 4A is a bar graph that shows the number of white blood cells(WBC) in each group of mice in the examples of the present invention; FIG. 4B is a bar graph that shows the number of platelets(PLT) in each group of mice in the examples of the present invention; FIG. 4C is a bar graph that shows the number of lymphocytes in each group of mice in the examples of the present invention; FIG. 4D is a bar graph that shows the number of neutrophil in each group of mice in the examples of the present invention; FIG. 4E is a bar graph that shows the number of eosinophil of mice of each group in the examples of the present invention; FIG. 4F shows the number of monocyte of mice in each group in the examples of the invention.

FIG. 5A is a bar graph that shows the number of red blood cells(RBC) in each group of mice in the examples of the present invention; FIG. 5B is a bar graph that shows the comparison of the hematocrit(HCT) of each group in the examples of the present invention; FIG. 5C is a bar graph that shows the mean corpuscular hemoglobin(MCH) in each group in the examples of the present invention; FIG. 5D is a bar graph that shows the mean corpuscular-hemoglobin concentration(MCHC) in each group of mice in examples of the present invention.

FIG. 6A, FIG. 6B and FIG. 6C show the drug treatment schedule of 1st group to 9st group in part II examples.

FIG. 7A shows the tumor volume of mice in group 2, group 3 and group4; FIG. 7B shows the tumor weight of mice in group 2, group 3 and group4. FIG. 8A shows the tumor volume of mice in group 2, group 8 and group 9; FIG. 8B shows the tumor weight of mice in group 2, group 8 and group 9.

FIG. 9A shows the tumor volume of mice in group 2, group 5, group 6 and group 7; FIG. 9B shows the tumor weight of mice in group 2, group 5, group 6 and group 7.

FIG. 10 shows the expression of IL-8 in group 2, group 3 and group 4.

FIG. 11 shows the expression of IL-8 in group 2, group 8 and group 9.

FIG. 12 shows the expression of IL-8 in group 2, group 5, group 6 and group 7.

### DETAILED DESCRIPTION OF THE INVENTION

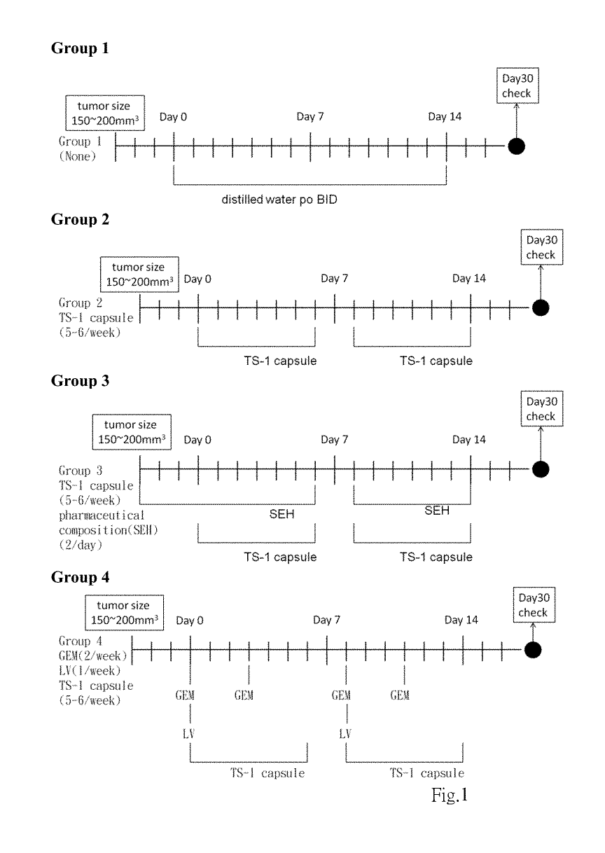

### Part I Shows the Examples 1 to Examples 7 and the FIG. 1 to FIG. 5D.

### The Experiments are Carried Out by Development Center for Biotechnology

### Example 1. The Preparation of the Presented Pharmaceutical Composition

First, the condensed solution was prepared by the materials of traditional chinese medicine list in Table 1. The preparation process comprising: (1) six times more amount of water was added into the 360 grams of traditional chinese medicine to make a 2160 mL solution; (2) the 2160 ml solution was condensed as 1200 mL by reduce pressure under high temperature (about 70° C.); (3) kept the condensed solution stable and cool, and removed 5% precipitate after two days to obtain a condensed solution.

TABLE 1
The traditional Chinese medicine of the pharmaceutical
composition of the present invention

| items | Chinese medicine raw materials | weight | |
|---|---|---|---|
| mushroom | Phellinus linteus | 30 | g |
| | Ganoderma lucidum | 20 | g |
| | Agaricus subrufescens | 20 | g |
| | Antrodia cinnamomea | 5 | g |
| | Caterpillar fungus | 5 | g |
| rhizome | Rhizoma polygonati | 20 | g |
| | Astragalus | 10 | g |
| | Salvia miltiorrhiza | 10 | g |
| | Codonopsis pilosula | 10 | g |
| | Hedyotis diffusa | 15 | g |
| | Eucommia ulmoides | 15 | g |
| | Atractylodes macrocephala | 10 | g |
| | Radix trichosanthis | 10 | g |
| | Eleutherococcus senticosus | 10 | g |
| | Ophiopogon japonicus | 10 | g |
| | Rhodiola | 10 | g |
| | Peeled Licorice | 3g | g |
| fruit | Job's Tears | 15 | g |
| | Ligustrum lucidum | 10 | g |
| | Schisandra chinensis | 10 | g |
| | Germinated brown rice | 12 | g |
| | Lotus seed | 10 | g |
| | Black sesame | 10 | g |
| | Corn Silk | 10 | g |
| | siraitia grosvenorii | 10 | g |
| | Red Grape Skin Extract Powder | 2 | g |
| leaf | Spinach | 10 | g |
| | Germinated Broccoli | 10 | g |
| | Papaya leaf | 10 | g |
| | Lotus leaf | 8 | g |
| flower | Chrysanthemum | 10 | g |
| alage | Seaweed | 10 | g |

Then, an energy-rich liquid, a salt-rich liquid, an assist agent and an anti-oxide agent were added sequentially into above mentioned 200 mL condensed solution which contains 60 grams of solid precipitate. After mixing, the solution was fermented at room temperature (25□) for 3-7 days. Preferably, fermentation is conduct 3 days. Said energy-rich liquid comprising 5 mg of ferric chloride (FeCl₃) and 200 ml of distilled water. Said salt-rich liquid comprising 125 grams of deep sea salt, 30 grams of magnesium chloride, 20 mL brine, 1.1 grams of calcium chloride (CaCl₂) and 0.55 grams of potassium chloride (KCL). Said assist agent comprising 20 grams of citric acid, 20 grams of selenium yeast, 10% concentration 300 mg of coenzyme Q10 and 3 grams of vitamin C. Said antioxidant comprising 70% concentration 10 grams of chitin oligosaccharides, 98% concentration 5 grams of glutathione, 80% concentration 1 g of pine bark and 80% concentration 1 gram of fucoidan. The antioxidant would have synergic effect to enhance the immunity.

The distilled water was added into the above-mentioned fermented solution to make a total volume of solution is 500 mL. After stirring, kept solution stable for 1 day and then centrifuged at 1500 rpm for 15 minutes. The supernatant is the pharmaceutical compositions of the present invention, which contains 25% deep sea salt, 6% magnesium chloride. The pH value of the pharmaceutical composition is 1.5±0.3 in order to maintain the product stability.

The pharmaceutical composition of the present invention is administered to a subject, wherein the individual is selected from the group consisting of a mammal selected from the group consisting of human, cow, pig, sheep and mouse.

The mice used in the experiments of the present invention are severe combined immunodeficiency mice (SCID mice), which white blood cells is between 1500-4500(cell/cu mm) compared with the white blood cells in normal mice is between 6000-15000 (cell/cu mm), thus the transplanted human cancer cells would definitely grow rapidly in these mice.

### Example 2. Seven Experimental Groups and the Preparation Thereof of the Present Invention

Cells Culture of MIA PaCa-2

MIA PaCa-2 (human pancreatic cancer) cell lines were cultured in Dulbecco's Medium Eagle Medium (DMEM) (Gibco; catalog: 11995-065). The medium contained 10% FBS (Gibco; catalog: 26140) and 2.5% HBS in a 5% CO², 37° C. incubator. When cell growth is about 80% full, suck out old cell culture medium, add cells with 0.05% Trypsin-EDTA (Gibco; catalog: 25300-062) and add fresh cell culture medium for cell subculture. Before MIA PaCa-2 cells were subcutaneously implanted, cells growing at log phase were removed and counted, and 1×10⁸ cells/mL were transplanted into DMEM medium without FBS and added with an equal volume of Matrigel solution (BD Falcons 354234, USA) to a final cell concentration of 5×10⁷/mL. MIA PaCa-2 cells were subcutaneously implanted.

8 weeks old CB-17 SCID mice (SCID mice: immunodeficient mice) is weighing 24.2±1.4 g, were subcutaneously inoculated with 100 μl (5×10 6/mouse) MIA PaCa-2 human pancreatic cancer cells that cause pancreatic cancer on the first day. Due to the rapid deterioration of pancreatic cancer, when the tumor volume to grow to 150-200 mm³, the male mice will be divided into the following seven groups, each group of 4 to 8, began a different course of drug treatment for 2 weeks, and then observe Mice were bled for two weeks and blood tests were performed on the 30th day.

### The Administration Method of the Pharmaceutical Composition of the Invention to an Individual

The pharmaceutical composition of the present invention (medium dosage) was diluted 1000-fold with high temperature and sterilized water and administered orally. Each mouse was given 417 ul each time (the dose of this mouse was 0.83 ul/mice: 1,000 times diluted with water, the volume of 833 ul; so BID (PO)=417 ul/times.), twice daily, 6 days a week (Monday to Saturday). However, if the pharmaceutical composition (medium dose) of the present invention is combined with the clinical anticancer drug TS-1, the pharmaceutical composition of the present invention (medium dose) is administered for 2 hours and then administered the TS-1 or the pharmaceutical composition of the present invention (medium dose). If the clinical anticancer drug is combined with other clinical anti-cancer drugs, the pharmaceutical composition of the present invention (medium dose) was not administered on that day. In addition, TS-1 capsule, leucovorin Calcium Injection, Fluoro-Uracil Germany, anticancer intravenous concentrate (CAMPTO Conc. Solution For IV Infusion), Gemcitabine and other commercially available cancer drug combinations were used in the experiments. Eschwein capsule referred to as TS-1 (Eschwesen capsule also known as TS-1 capsule, It is Tegafur, Gimeracil, Oteracil potassium combination of three components), Dai Libu Boning live Lin injection referred to as LV, favorable cancer referred to as 5-FU, anticancer intravenous concentrate concentrate referred to as CPT11 and Jianze referred to as GEM hereinafter.

(Note: medical staff members will not be assigned to inject the pharmaceutical composition on Sunday because the reshuffle on Sundays. Therefore, it will be more special to administer the pharmaceutical composition on Saturday (1.66 ul/mice/time on Saturday Afternoon. Each)), try to extend the efficacy until Sunday)

The seven groups of mice were as follows:

TABLE 2
Groups and the drugs administered

| Groups | Drugs administered |
|---|---|
| Group 1 | None |
| Group 2 | TS-1 capsule |
| Group 3 | TS-1 capsule + the pharmaceutical composition of the present |
| | invention |
| Group 4 | GEM + LV + TS-1 capsule |
| Group 5 | GEM + LV + TS-1 capsule + the pharmaceutical composition |
| | of the present invention |
| Group 6 | ONIVYDE (micro-lipidated CPT11) + LV + 5-FU (combined |
| | the treatment of micro-lipidated CPT11, LV and 5-FU) |
| Group 7 | ONIVYDE + LV + 5-FU + the pharmaceutical composition |
| | of the present invention |

### Group 1: None

As shown in FIG. 1 (1), after SCID immunodeficient mice were inoculated with human MIA-Paca 2 pancreatic cancer cells, mice with tumor size ranging from 150 mm³ to 200 mm³ were selected and started three days later daily cast distilled water twice (oral administration) for a total of 14 days, and checked the blood composition changes from blood test in the first 30 days.

### Group 2: TS-1 Capsule

As shown in FIG. 1 (2), after SCID immunodeficient mice were inoculated with MIA-Paca 2 pancreatic cancer cells, mice with tumor size ranging from 150 mm³ to 200 mm³ were selected and started three days later TS-1 was administered daily (administered orally at a concentration of 15 mpk) for a total of 14 days. The number of injections per week was 5-6. The behavior of the rats was observed and recorded for two weeks, and checked the blood composition changes from blood test in the first 30 days.

### Group 3: TS-1 Capsule+the Pharmaceutical Composition of the Present Invention

As shown in FIG. 1 (3), after SCID immunodeficient mice were inoculated with MIA-Paca 2 pancreatic cancer cells, mice with tumor size ranging from 150 mm³ to 200 mm³ were selected and started three days test, the pharmaceutical composition of the present invention was administered daily (administered orally at a concentration of 15 mpk) for a total of 14 days. Two hours after the administration of the pharmaceutical composition of the present invention on day 0, TS-1 chemotherapeutic drug (administered orally at a dose of 15 mpk) was administered for a total of two weeks. The number of TS1-injections per week was 5-6. The behavior of the mice was observed and recorded for two weeks, and checked the blood composition changes from blood test in the first 30 days.

### Group 4: GEM+LV+TS-1 Capsule

As shown in FIG. 1 (4), after SCID immunodeficient mice were inoculated with human MIA-Paca 2 pancreatic cancer cells, mice with tumor size ranging from 150 mm³ to 200 mm³ were selected, and LV was administered on day 0 and day 8 (IP intraperitoneal injection at 90 mpk), GEM was administered on day 0, day 3, day 8, and day 11 (IP ip=100 mpk), and TS-1 was administered (administered orally at a dose of 15 mpk) for two consecutive weeks. Wherein GEM was administered 2 times a week, LV was administered once a week, LV was administered for 5 to 6 times weekly, and the time interval of each drug injection is 1 hour. The behavior of the rats was observed and recorded for two weeks, and checked the blood composition changes from blood test in the first 30 days.

### Group 5: GEM+LV+TS-1 Capsule+the Pharmaceutical Composition of the Present Invention

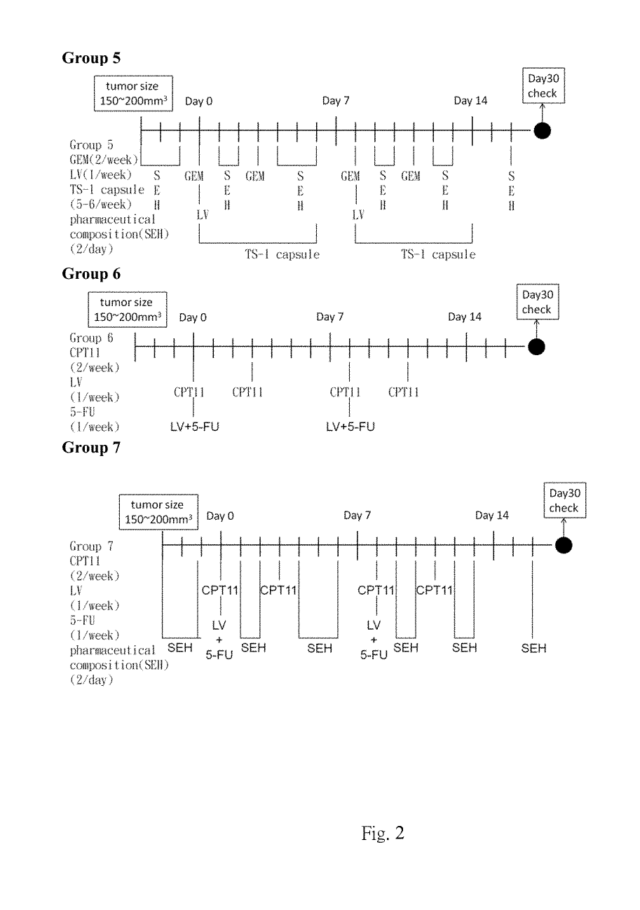

As shown in FIG. 2 (5), after SCID immunodeficient mice were inoculated with human MIA-Paca 2 pancreatic cancer cells, mice with tumor size ranging from 150 mm³ to 200 mm³ were selected, and started three days test. The present pharmaceutical compositions were administered twice a day and LV (IP administration at dose of 90 mpk) was administered on day 0, day 8, GEM (IP administration at dose of 90 mpk) was administered on day 0, day 3, day 8, day 11, TS-1 administered 2 hours after the pharmaceutical composition of the present invention was administered (administered orally at a dose of 15 mpk) for total of two weeks. Wherein the administrations of GEM was 2 times weekly, LV administration was 1 time weekly, and administrations of TS-1 was 5-6 times weekly, the time interval of each drug injection is 1 hour). The behavior of the mice was observed and recorded for two weeks, and checked the blood composition changes from blood test in the first 30 days.

### Group 6: ONIVYDE (Micro-Lipidated CPT11)+LV+5-FU

### (Combined the Treatment of Micro-Lipidated CPT11, LV and 5-FU)

As shown in FIG. 2 (6), after SCID immunodeficient mice were inoculated with human MIA-Paca 2 pancreatic cancer cells, mice with tumor size ranging from 150 mm³ to 200 mm³ were selected, and started three days test. LV (IP injection at dose of 90 mpk) and 5-FU (IV injection at dose of 30 mpk) was administered on day 0, day 8, CPT 11 (IP injection at dose of 90 mpk) was administered on day 0, day 3, day 8, day 11. CPT 11 administration was 2 times weekly, LV administration was 1 time weekly, and administrations of 5-FU was 1 time weekly, the time interval of each drug injection is 1 hour. The behavior of the mice was observed and recorded for two weeks, and checked the blood composition changes from blood test in the first 30 days.

### Group 7: ONIVYDE (Micro-Lipidated CPT11)+LV+5-FU+the Pharmaceutical Composition of the Present Invention

As shown in FIG. 2 (7), after the SCID immunodeficient mice were inoculated to human MIA-Paca2 pancreatic cancer tumor cells, mice with tumor sizes ranging from 150 mm³ to 200 mm³ were selected, and the present pharmaceutical compositions was administered daily on the first 3 days (day-3). The composition was given twice a day, and LV (IP injection at dose of 90 mpk) and 5-FU (IV injection at dose of 30 mpk) were administered on day 0 and day 8, the lipidated CPT11(IP injection at dose of 90 mpk) was administered and on day 0, day 3, day 8, day 11. the lipidated CPT11 administration was 2 times weekly, LV administration was 1 time weekly, and administrations of 5-FU was 1 time weekly, the time interval of each drug injection is 1 hour. The behavior of the mice was observed and recorded for two weeks, and checked the blood composition changes from blood test in the first 30 days.

### Example 3. The Pharmaceutical Composition of the Present Invention on the Growth of Pancreatic Tumors

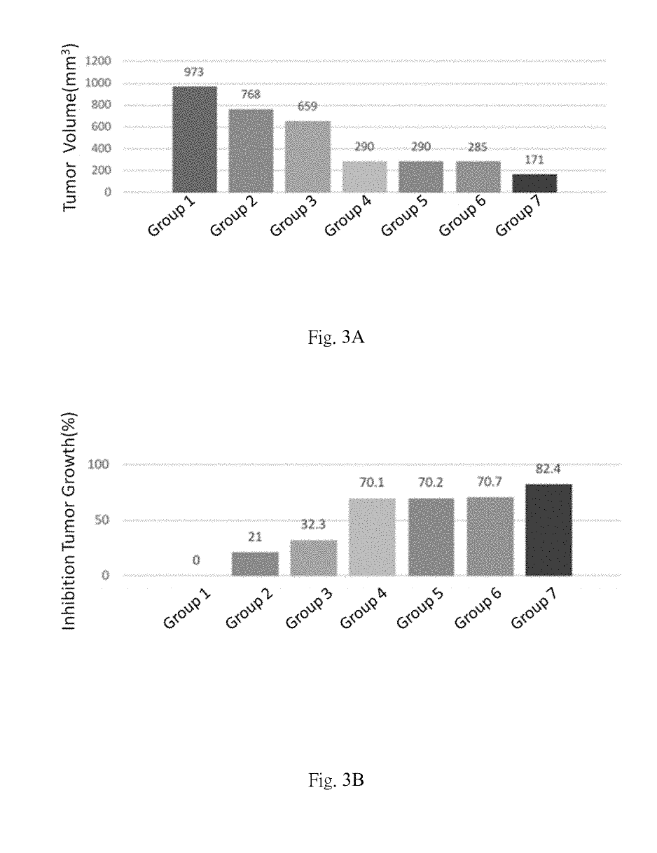

On day 30, in each group of example 2, the tumor mass was removed and its weight measured. Please refer to FIG. 3A-3B, the results showed the inhibitory effect on pancreatic tumor in all groups from group 2 to the group 7. In comparison of the group 2 and the group 3, the tumor inhibition rate increased from 21% in the group 2 (TS1 group) to 32.3% in the group 3(TS1+the pharmaceutical composition of present invention). The tumor inhibition rate was no significant difference between the group 4 and the group 5, the group 4 (LV+GEM+TS1 group) and the group 5 (LV+GEM+TS1+the pharmaceutical composition of present invention). In comparison of the Group 6 (ONIVYDE+LV+5-FU) and the group 7 (ONIVYDE+LV+5-FU+the pharmaceutical composition of the present invention), the tumor inhibition rate of mice was 70.7% in the group 6 to 82.4% in the group 7 (ONIVYDE+LV+5-FU+the pharmaceutical composition of the present invention), which was increased 17%.

### Example 4. The Effect of the Presented Pharmaceutical Compositions on Immune System

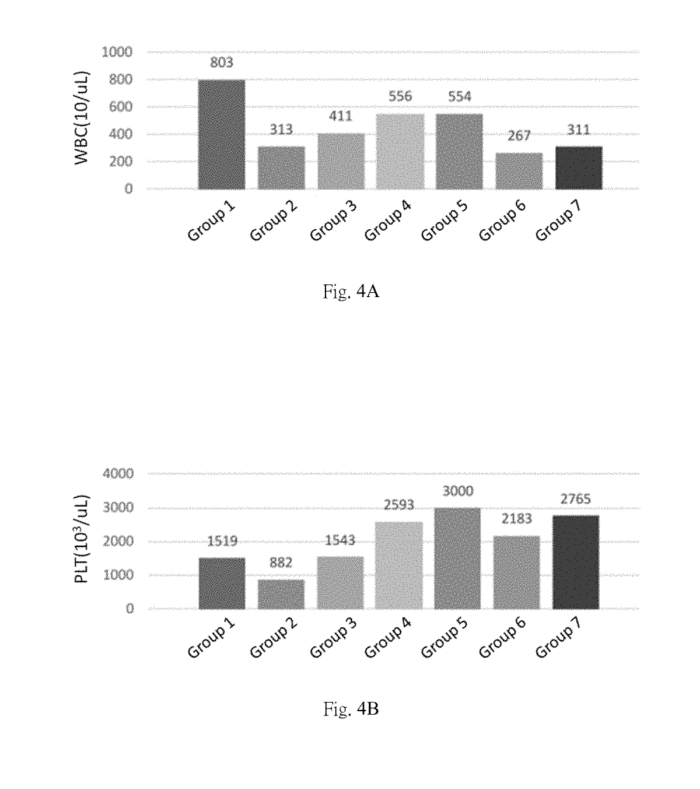

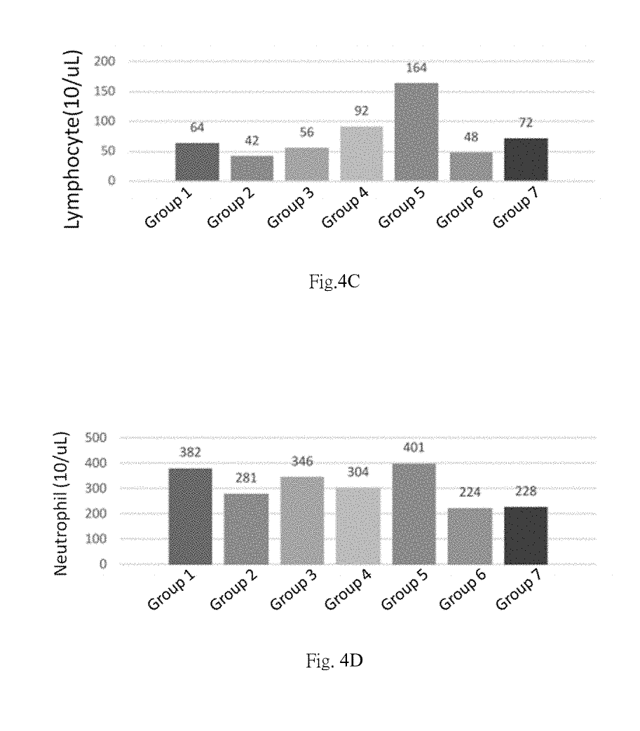

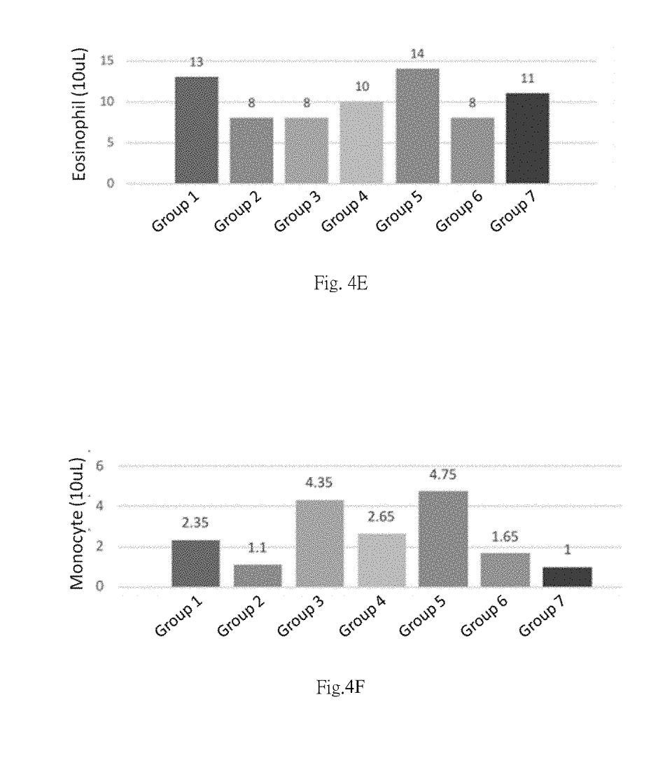

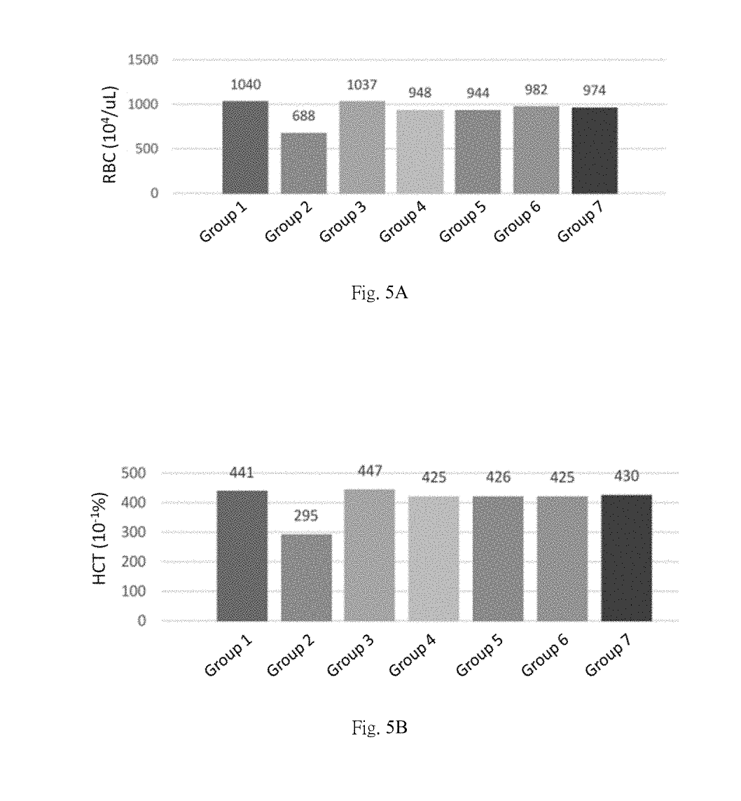

It is well known that the immune cells and cytokines are involved in inhibit cancer growth. Immune cells are white blood cells, including lymphocytes, mononuclear and granular balls, etc., are important tissue for immune system, which can help the body to resist infectious diseases and foreign objects. Cytokines play a signal role in cell message delivery. Cytokines can be released by different cells and deliver different cell messages that alter the function of the cells. To understand the effect of presented pharmaceutical compositions on immune system and cytokines, the blood of each group in example 2 was collected at day 30 after administered drugs to analyze the numbers of immune cells and cytokines. Please refer to FIG. 4A-4F.

Please refer to FIG. 4A, the results showed that the number of white blood cells in the group 3 (TS1+the pharmaceutical composition of present invention), the group 7 (ONIVYDE+LV+5-FU+the pharmaceutical composition of present invention) was more than, the number of platelets than the group 2 (TS-1), the group 6 (ONIVYDE+LV+5-FU), especially the number of white blood cells in the group 7 (ONIVYDE+LV+5-FU+the pharmaceutical composition of present invention) is more than in the group 6 (ONIVYDE+LV+5-FU) by 17% more. Please refer to FIG. 4B, the results showed that the number of platelets in the group 3, the group 5, the group 7 was more than in the group 2, the group 4, the group 6 more, especially the number of platelets in the group 7 was more than in the sixth group by 27% more. Please refer to FIG. 4C, the results showed that the number of lymphocytes in the group 3, the group 5, the group 7, are more than in the group 2, the group 4, the sixth group more, in particular, the number of lymphocytes the seventh group was 50% more than the group 6. Please refer to FIG. 4D, the results showed that the number of neutrophils in the group 3, the group 5, the group 7, was more than the group 2, the group 4, the group 6. Please refer to FIG. 4E, the results show that the number of acidic cells in the group 5, the group 7 was more than the group 4, the group 6. Please refer to FIG. 4F, the results showed that the number of acidic cells in the group 3, the group 5 was more than the group 2 and the group 4.

Based on the above experimental results, the pharmaceutical composition of the present invention simultaneously promotes the proliferation of white blood cells in the blood (FIG. 4A), bringing the number of white blood cells in the experimental group close to the medium-high standard of SCID mice. The number of leukocytes in SCID mice was in the range of 1,500-4,500 (cell/cu mm), while the number of normal mouse white blood cells was in the range of 6,000-15,000 (cell/cu mm).

### Example 5. The Impact of the Pharmaceutical Composition of the Present Invention on Red Blood Cells, Hematocrit, Mean Corpuscular Hemoglobin, Mean Corpuscular-Hemoglobin Concentration

Based on the clinical trial of TS-1, the important clinical side effects include erythrocyte decrease, leukopenia decrease, hematocrit decrease, neutropenia decrease, heme decrease, loss of appetite, nausea, tiredness, pigmentation disorders, diarrhea, stomatitis, rash, increased ALT, platelet decrease and other side effects. Therefore, the following experiments on the side effects of TS-1 by the pharmaceutical combination of the present invention will be performed.

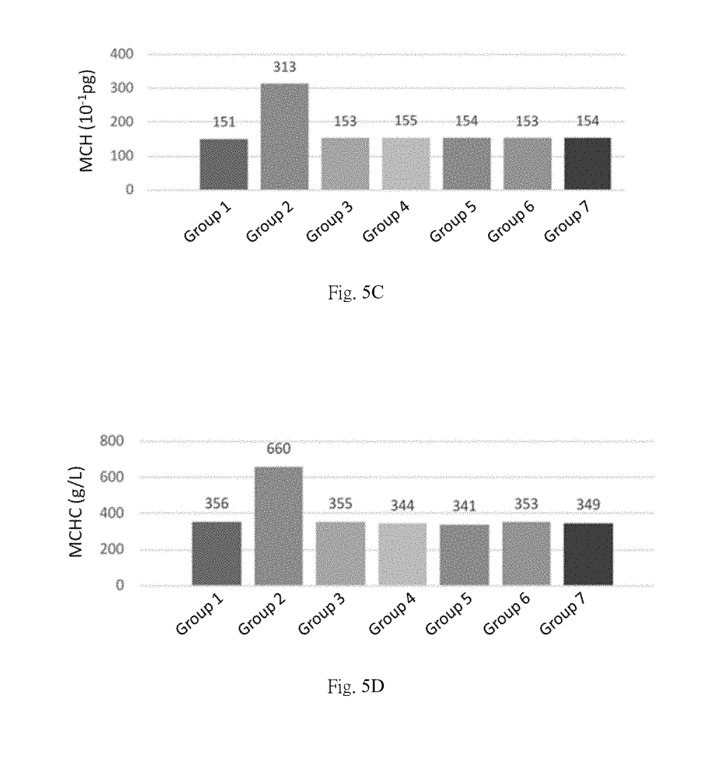

Please refer to FIG. 5A, 5B, 5C, 5D, the results showed that in comparison of the group 4 and the group 6, the number of red blood cells and hematocrit was less in the group 2, and the average amount of red blood cell hemoglobin, the average of mean corpuscular-hemoglobin were more. There were no significant differences in the numbers of red blood cells, hematocrit, mean corpuscular hemoglobin and mean corpuscular-hemoglobin concentration in the group 4 and group 6 compared with the group 1. Compared with the group 2, the number of red blood cells and hematocrit in the group 3 was increased and increased to similar number of the group 1. In addition, compared with the group 2, the mean corpuscular hemoglobin and the mean corpuscular-hemoglobin concentration of the group 3 was significantly lower, and the mean corpuscular-hemoglobin concentration was decreased to the similar concentration of group 1. However, there was no significant difference between the group 5 and the group 7 respectively compared with the group 4 and the group 6.

Chemotherapy with TS-1 caused abnormalities in red blood cells, hematocrit, mean corpuscular hemoglobin and the mean corpuscular-hemoglobin concentration in mice, whereas the pharmaceutical composition of the present invention has the effect of reducing side effects of TS-1, so that balance the number of red blood cells, hematocrit, mean corpuscular hemoglobin and the mean corpuscular-hemoglobin concentration.

### Example 6. The Effect of the Pharmaceutical Composition of the Present Invention on Enhancing the Treatment of Pancreatic Cancer

The tumor volume of the above seven groups of mice as shown in FIGS. 3A and 3B, wherein the results showed that:

In comparison of tumor volume of mice administered with TS-1 (Group 2) and with TS-1+the pharmaceutical composition of the present invention (Group 3) showed that administration of the TS-1+pharmaceutical composition of the present invention (Group 3) mice had smaller tumor volumes than mice that administered TS-1 (Group 2), indicating that the pharmaceutical composition of the present invention can potentiate the therapeutic effect of pancreatic cancer of TS-1.

However, there was no significant difference in the tumor volume between mice administered with LV+GEM+TS1 (Group 4) and vehicle+LV+GEM+TS1+(Group 5), indicating that the pharmaceutical compositions of the present invention failed to significantly potentiate the effect of the combination therapy of therapeutic drugs LV and GEM and TS1.

Next, the tumor volume of mice that was treated with ONIVYDE+LV+5-FU (Group 6) and ONIVYDE+LV+5-FU+the pharmaceutical composition of the present invention (Group 7) was compared. The tumor volume of mice that was administered with ONIVYDE+LV+5-FU+the pharmaceutical composition of the present invention (Group 7) is smaller than the mice administered ONIVYDE+LV+5-FU (Group 6), indicating that the pharmaceutical composition of the present invention can potentiate the therapeutic effect of ONIVYDE+LV+5-FU on pancreatic cancer.

### Example 7. The Effect of Pharmaceutical Composition of the Present Invention on Reducing Side Effects of the Chemotherapy Drugs

By comparing the number of lymphocytes in the blood of the mice in the seven groups, the effect of the pharmaceutical composition of the present invention on reducing the side effects of the chemotherapeutic drugs was investigated. The result was shown in FIG. 4C.

The number of lymphocytes in mice administered with TS-1 (Group 2) versus TS-1+administration of the pharmaceutical composition of the invention (Group 3) was compared. The number of lymphocytes in the mice treated with TS-1+the pharmaceutical composition of the present invention (Group 3) was more than that of mice administered with TS-1 (Group 2), which indicating that the pharmaceutical composition of the present invention can reduce the side effects of the chemotherapeutic agent TS-1.

Next, the number of lymphocytes in mice administered with LV+GEM+TS1 (Group 4) with LV+GEM+TS1+administered pharmaceutical compositions of the present invention (Group 5) was compared. The number of lymphocytes in the mice treated with LV+GEM+TS1 (Group 4) was more than that of mice administered with LV+GEM+TS1+administered pharmaceutical compositions of the present invention (Group 5), which indicating that the pharmaceutical composition of the present invention can reduce the side effects of the chemotherapeutic agent TS-1+GEM+TS-1.

Next, the number of lymphocytes in mice administered with ONIVYDE+LV+5-FU (Group 6) and ONIVYDE+LV+5-FU+the pharmaceutical composition of the present invention(Group 7) was compared. The number of lymphocytes in the mice treated with ONIVYDE+LV+5-FU+the pharmaceutical composition of the present invention (Group 7) was more than that of mice administered with ONIVYDE+LV+5-FU (Group 6), which indicating that the pharmaceutical composition of the present invention can reduce the side effects of the chemotherapeutic agent TS-1+GEM+TS-1.

The effects of the pharmaceutical composition of the present invention on the side effects of the chemotherapeutic drug TS-1 interfering with erythrocytes were investigated by comparing the numbers of red blood cells, hematocrit, heme measurement, and heme concentration in the blood of the eight groups of mice. As shown in FIG. 5A, FIG. 5B, FIG. 5C and FIG. 5D, the amount of hematocrit, the hematocrit, the hematocrit of the red blood cells, the hematocrit of the hematocrit of the mice treated with TS-1 (Group 2) and the TS-1+pharmaceutical composition of the present invention (Group 3). The results showed abnormal erythrocytes in the blood of mice treated with TS-1 (Group 2), and erythrocyte abnormalities in the blood of mice administered TS-1+the pharmaceutical composition of the present invention (Group 3) have been restored, Indicating that the pharmaceutical composition of the present invention can reduce the side effects of the chemotherapeutic agent TS-1.

In summary, the effect of pharmaceutical composition of the present invention has been confirmed by animal experiments conducted by the Biotechnology Development Center under the Ministry of Economic Affairs. MIA-PaCa 2 pancreatic cancer cells inoculated mice were administered with chemotherapy for 2 weeks and observed for two weeks. The pharmaceutical compositions of the present invention were found to be compatible with current chemotherapeutic drugs such as TS-1 capsule, leucovorin Calcium Injection, Fluoro-Uracil Germany, CAMPTO Conc. Solution For IV Infusion, Gemcitabine, or a combination of all the drugs above, not only make the tumor smaller than chemotherapy alone, but also increase the number of white blood cells, platelets, lymphocytes, neutral spheres, eosinophils, and the balance of hematocrit (HCT), mean corpuscular hemoglobin (MCHC), mean corpuscular-hemoglobin concentration (MCHC).

The pharmaceutical compositions of the present invention may have different effects on different chemotherapeutic agents. (1) Having the effect of reducing side effects and strengthening of chemotherapy drugs treating pancreatic cancer such as TS-1 and ONIVYDE+LV+5-FU. (2) Reducing the side effects of combined chemotherapy drugs of LV, GEM and TS1.

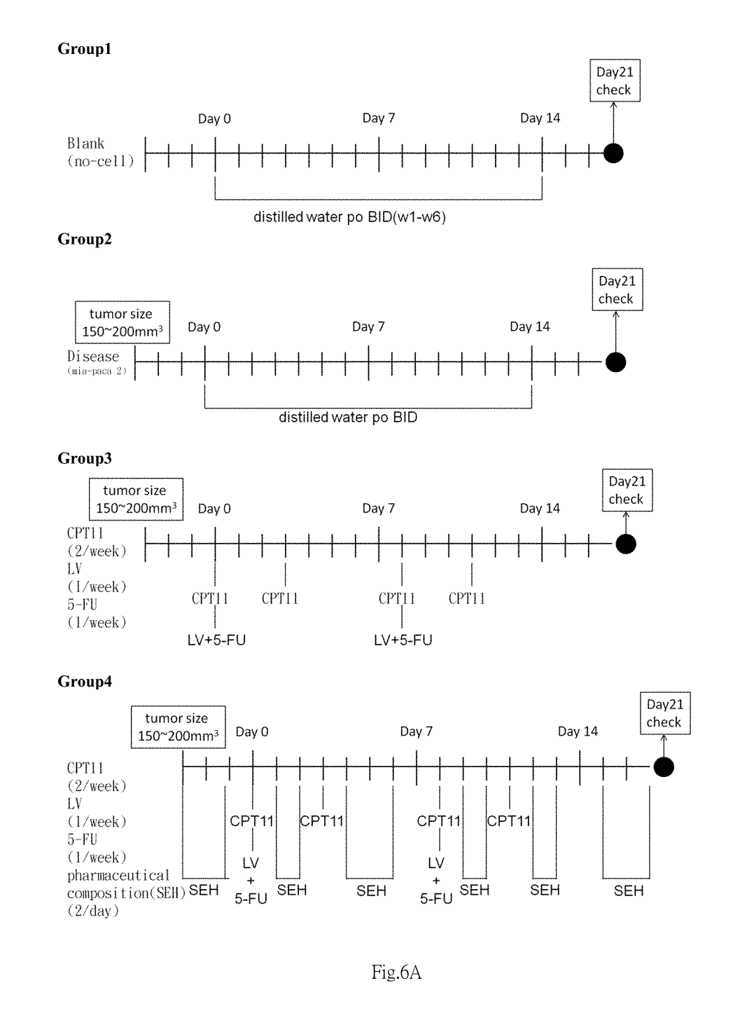

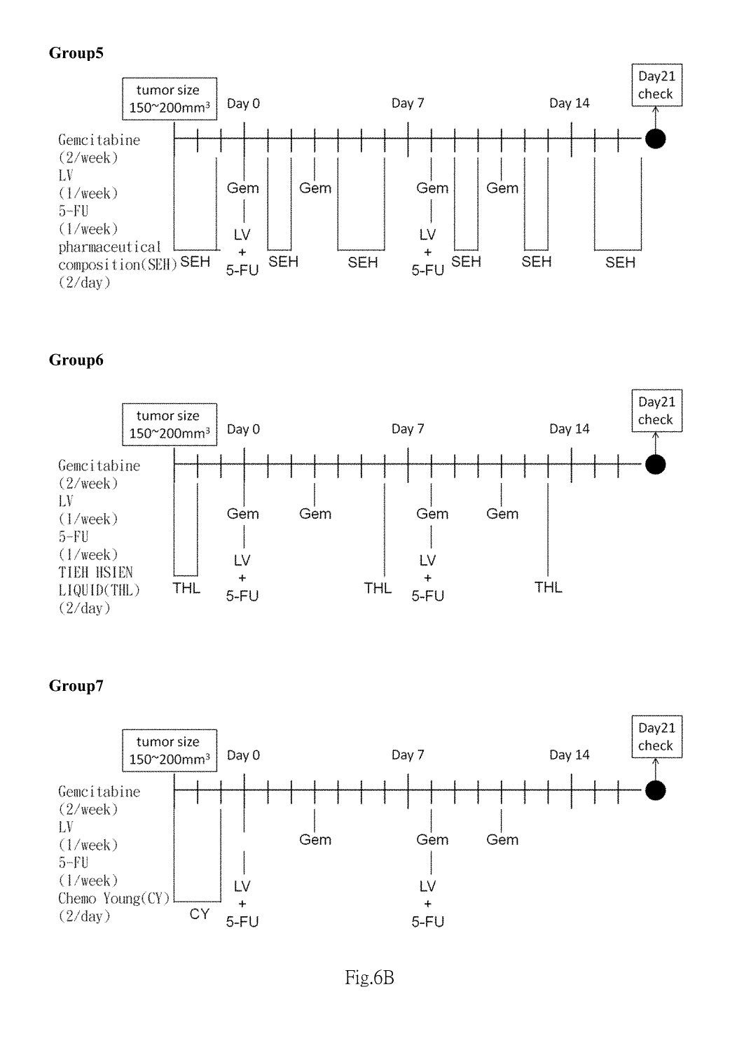

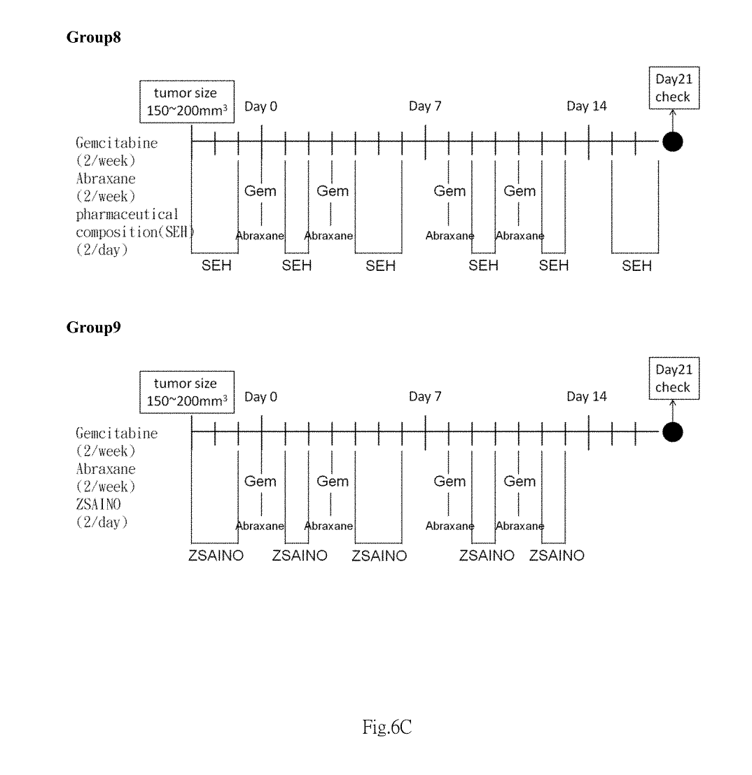

### Part II Shows the Example 8 to Example 9 and the FIG. 6A to FIG. 12

Cells Culture of MIA PaCa-2

MIA PaCa-2 (human pancreatic cancer) cell lines were cultured in Dulbecco's Medium Eagle Medium (DMEM) (Gibco; catalog: 11995-065). The medium contained 10% FBS (Gibco; catalog: 26140) and 2.5% HBS in a 5% CO², 37° C. incubator. When cell growth is about 80% full, suck out old cell culture medium, add 0.05% Trypsin-EDTA (Gibco; catalog: 25300-062) to detach cells and add fresh cell culture medium for cell subculture. Before MIA PaCa-2 cells were subcutaneously implanted, cells growing at log phase were removed and counted, and 1×10⁸ cells/mL were transplanted into DMEM medium without FBS and added with an equal volume of Matrigel solution (BD Falcons 354234, USA) to a final cell concentration of 5×10⁷/mL. MIA PaCa-2 cells were subcutaneously implanted.

Experimental Animals:

1. Animal breed: Mice
2. Animal strain: C.B-17 SCID
3. Animal sex: public
4. Animal age: 8 weeks old

### 5. Animal Source: Lesco Biotech

6. Animal quarantine: Animals are quarantined by animal veterinarians for one week before entering the breeding room.
7. Animal weight: 24.5±1.4 g
8. Animal number: Animals are numbered with ear tags, and each breeding cage is accompanied by an identification card, which records the cage number, group, gender and animal number.
9. Number of animals: Animals are divided into 9 groups, 4 in each group, a total of 36
10. Feeding environment: The breeding room is set at a temperature of 20-26° C., humidity of 50±10% and 12 hours of light, 12 hours of dark light cycle. Feed (Purina Certified Rodent Chow) and water unlimited supply.
11. The number of animal experiments and animal room test methods were reviewed by the National Taiwan Laboratory Animal Care and Use Committee. The animal experiment application form and consent form number is: 106 Empirical Word No. 00099.

Reagents and Instruments

1. Weight device

### 2. Vernier (Series No. 500, Mitutoyo Corp.)

3. Automatic blood cell analyzer XT1800iv (Sysmex)
4. Liposome Kit: Lipid mixtures (L4395; SIGMA-ALDRICH)

### 5. Milliplex MCYTOMAG-70K Kit (Millipore)

This animal model was implanted with 100 μl (5×106/mouse) MIA PaCa-2 cells subcutaneously on the first day using 8 weeks of CB-17 SCID mice, and divided into 9 groups on the 8th day when the tumor volume averaged 151.7±18.5 mm3 and the test substance was started to be administered orally. The 9 groups: blank group(group 1); disease group (non-drug) (group 2); experimental group (2 test substances and 4 clinical anticancer drugs divided into 7 groups in different combinations)(group3˜group9).

Test Substance

1. The pharmaceutical composition of the present invention (SEH): the pharmaceutical composition of the present invention, given by BID (twice a day).
2. Tieh Hsien Liquid (THL): Main ingredients of THL include ginseng, caterpillar fungus, astragalus, licorice, Chinese yam, pearl, wolfberry seeds, lingzhi mushroom, ligustrum seed, barbat skullcap herb.
3. Chemo young (CY): organic soybean and probiotics fermentation products
4. ZSAINO: contains unique patented ingredient Antroquinonol. Antroquinonol is the pure compound extracted from Antrodia camphorata. Antroquinonol is patented by Golden Biotech and helps support the immune system.

### Test Design

1. Grouping: The 8 week old C.B-17 SCID mice were divided into 9 groups of 4 mice each with a total of 36. The 9 groups of treatment were shown as FIG. 6.
2. The pharmaceutical composition of the present invention was diluted the stock solution by 1000, 700 or 50 times, and configure it into four doses: low, medium, high and super high. They were administered orally, and each mouse was given 250 ul, 417 ul or 585 ul each time, twice a day, 6 days a week (Monday to Saturday), and the course of treatment was 3 weeks (17 days). If the pharmaceutical composition of the present is combined with clinical anticancer drug therapy, the pharmaceutical composition of the present will not be used on the same day as the clinical anticancer drug. If the pharmaceutical composition of the present is to be used on the same day as the clinical anticancer drug, SEH997 will be given first, and then the clinical anticancer drug will be given after 2 hours.

The nine groups of mice were as follows:

TABLE 3
Groups and the drugs administered

| Groups | Drugs administered |
|---|---|
| Group 1 | Blank(non-cell) |
| Group 2 | Disease(MIA-Paca2) |
| Group 3 | LV + 5FU + ONIVYDE |
| Group 4 | LV + 5FU + ONIVYDE + the pharmaceutical composition |
| | of the present invention (SEH997) |
| Group 5 | LV + 5FU + GEM + the pharmaceutical composition of |
| | the present invention (SEH) |
| Group 6 | LV + 5FU + GEM + Tieh Hsien Liquid (THL) |
| Group 7 | LV + 5FU + GEM + Chemo young (CY) |
| Group 8 | GEM + Abrax + the pharmaceutical composition of the present |
| | invention (SEH) |
| Group 9 | GEM + Abrax + ZSAINO |

### Group 1: Blank (Non-Cell)

As shown in FIG. 6 (1), the 8 week old C.B-17 SCID mice were treated with distilled water twice (oral administration) for a total of 14 days, and observed for one week from day 15, then sacrificed on day 21.

### Group 2: Disease (MIA-Paca 2)

As shown in FIG. 6 (2), the 8 week old C.B-17 SCID mice were inoculated with MIA-Paca 2 pancreatic cancer cells, then treated with distilled water twice (oral administration) for a total of 14 days. The behavior of the mice was observed and recorded for one week from day 15, then the mice were sacrificed on day 21.

### Group 3: LV+5-FU+ONIVYDE

As shown in FIG. 6. (3), after SCID immunodeficient mice were inoculated with human MIA-Paca 2 pancreatic cancer cells, mice with tumor size ranging from 150 mm³ to 200 mm³ were selected, and started three days test. The present pharmaceutical compositions were administered twice a day and LV (IP injection at dose of 90 mpk) and 5-FU (IV injection at dose of 30 mpk) was administered on day 0, day 8; and CPT 11 (IP injection at dose of 90 mpk) was administered on day 0, day 3, day 8, day 11. CPT 11 administration was 2 times weekly, LV administration was 1 time weekly, and administrations of 5-FU was 1 time weekly, the time interval of each drug injection is 1 hour. The behavior of the mice was observed and recorded for one week from day 15, and, then the mice were sacrificed on day 21.

### Group 4: LV+5-FU+ONIVYDE+the Pharmaceutical Composition of the Present Invention

As shown in FIG. 6 (4), after SCID immunodeficient mice were inoculated with human MIA-Paca 2 pancreatic cancer cells, mice with tumor size ranging from 150 mm³ to 200 mm³ were selected. The present pharmaceutical compositions were administered twice a day as two different dosages: PO at dose of 0.5 ul on day −3, day −2, day −1, day 1, day 2, day 5, day 9, day 10, day 12, and day 15 to day 21; PO at dose of 1 ul on day 6, day 13; LV (IP injection at dose of 90 mpk) and 5-FU (IV injection at dose of 30 mpk) was administered on day 0, day 8, CPT 11 (IP injection at dose of 90 mpk) was administered on day 0, day 3, day 8, day 11. CPT 11 administration was 2 times weekly, LV administration was 1 time weekly, and administrations of 5-FU was 1 time weekly, the time interval of each drug injection is 1 hour. The behavior of the mice was observed and recorded for one week from day 15, and then the mice were sacrificed on day 21.

### Group 5: LV+5-FU+GEM+the Pharmaceutical Composition of the Present Invention

As shown in FIG. 6. (5), after SCID immunodeficient mice were inoculated with human MIA-Paca 2 pancreatic cancer cells, mice with tumor size ranging from 150 mm³ to 200 mm³ were selected, and started three days test. The present pharmaceutical compositions were administered twice a day as two different dosages: PO at dose of 0.5 ul on day −3, day −2, day −1, day 1, day 2, day 5, day 9, day 10, day 12, and day 15 to day 21; PO at dose of 1 ul on day 6, day 13; LV (IP injection at dose of 90 mpk) and 5-FU (IV injection at dose of 30 mpk) was administered on day 0, day 8; GEM (IP administration at dose of 100 mpk) was administered on day 0, day 3, day 8, day 11. The time interval of each drug injection is 1 hour. The behavior of the mice was observed and recorded for one week from day 15, and then the mice were sacrificed on day 21.

### Group 6: LV+5-FU+GEM+THL

As shown in FIG. 6. (6), after SCID immunodeficient mice were inoculated with human MIA-Paca 2 pancreatic cancer cells, mice with tumor size ranging from 150 mm³ to 200 mm³ were selected, and started three days test. LV (IP injection at dose of 90 mpk) and 5-FU (IV injection at dose of 30 mpk) was administered on day 0, day 8; GEM (IP administration at dose of 100 mpk) was administered on day 0, day 3, day 8, day 11; Tieh Hsien Liquid (THL) were administered twice a day as two different dosages: PO at dose of 12.3 ml/kg on day −3; PO at dose of 24.6 ml/kg on day −2, day 6, day 13; LV administration was 1 time weekly, and administrations of 5-FU was 1 time weekly, the time interval of each drug injection is 1 hour. The behavior of the mice was observed and recorded for one week from day 15, and then the mice were sacrificed on day 21.

### Group 7: LV+5-FU+GEM+CY

As shown in FIG. 6. (6), after SCID immunodeficient mice were inoculated with human MIA-Paca 2 pancreatic cancer cells, mice with tumor size ranging from 150 mm³ to 200 mm³ were selected, and started three days test. LV (IP injection at dose of 90 mpk) and 5-FU (IV injection at dose of 30 mpk) was administered on day 0, day 8; GEM (IP administration at dose of 100 mpk) was administered on day 0, day 3, day 8, day 11; Chemo young(CY) were administered twice a day as two different dosages: PO at dose of 1.64 ml/kg on day −3, day −2, day −1, day 1, day 2, day 4, day 9, day 10, day 12; PO at dose of 3.28 ml/kg on day 6, day 13; LV administration was 1 time weekly, and administrations of 5-FU was 1 time weekly, the time interval of each drug injection is 1 hour. The behavior of the mice was observed and recorded for one week from day 15, and then the mice were sacrificed on day 21.

### Group 8: GEM+Abrax+the Pharmaceutical Composition of the Present Invention

As shown in FIG. 6. (8), after SCID immunodeficient mice were inoculated with human MIA-Paca 2 pancreatic cancer cells, mice with tumor size ranging from 150 mm³ to 200 mm³ were selected, and started three days test. The present pharmaceutical compositions were administered twice a day as two different dosages: PO at dose of 0.5 ul on day −3, day −2, day −1, day 1, day 2, day 5, day 9, day 10, day 12, and day 15 to day 21; PO at dose of 1 ul on day 6, day 13; GEM (IP administration at dose of 100 mpk) and Abrax (IV administration at dose of 10 mpk) was administered on day 0, day 3, day 8, day 11. The time interval of each drug injection is 1 hour. The behavior of the mice was observed and recorded for one week from day 15, and then the mice were sacrificed on day 21.

### Group 9: GEM+Abrax+ZSAINO

As shown in FIG. 6. (9), after SCID immunodeficient mice were inoculated with human MIA-Paca 2 pancreatic cancer cells, mice with tumor size ranging from 150 mm³ to 200 mm³ were selected, and started three days test. ZSAINO (PO at dose of 110.7 mg/kg) on day −3, day −2, day −1, day 1, day 2, day 5, day 9, day 10, day 12, and day 15 to day 21; PO at dose of 1 ul on day 6, day 13; GEM (IP administration at dose of 100 mpk) and Abrax (IV administration at dose of 10 mpk) was administered on day 0, day 3, day 8, day 11. The time interval of each drug injection is 1 hour. The behavior of the mice was observed and recorded for one week from day 15, and then the mice were sacrificed on day 21.

Observation Index

### 1. Tumor Growth Situation:

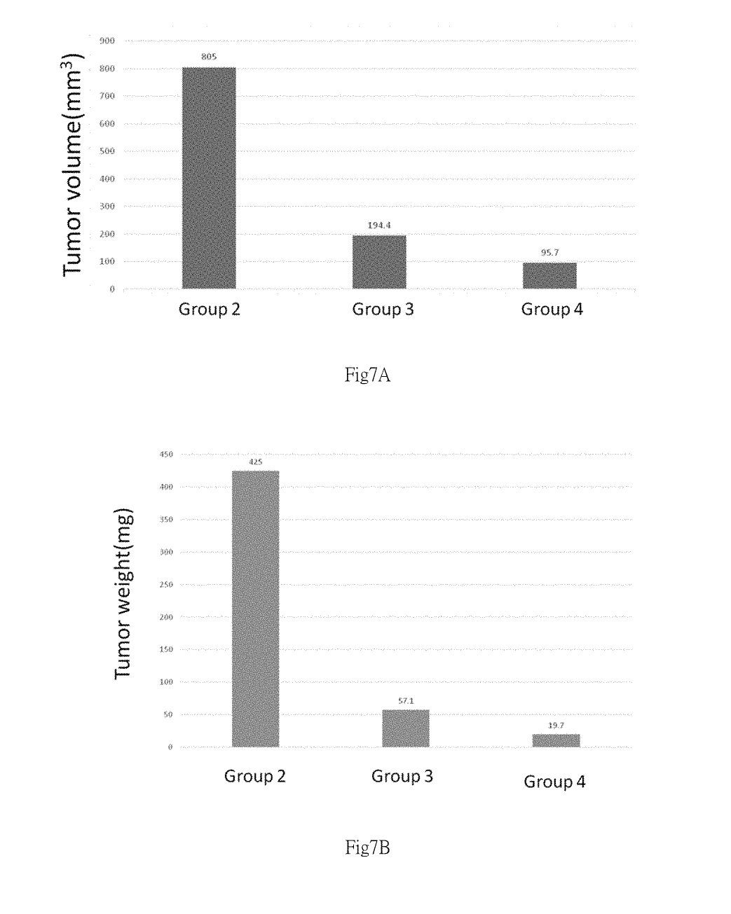

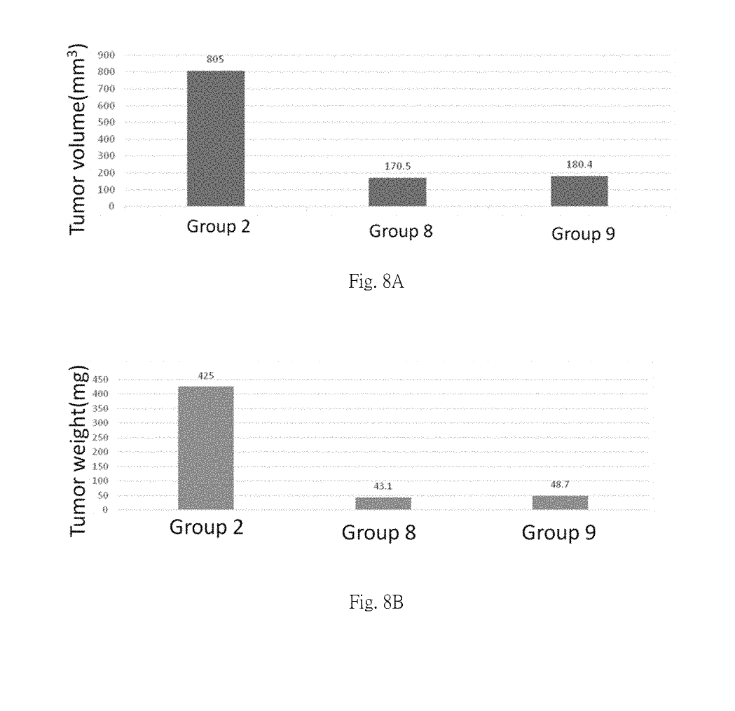

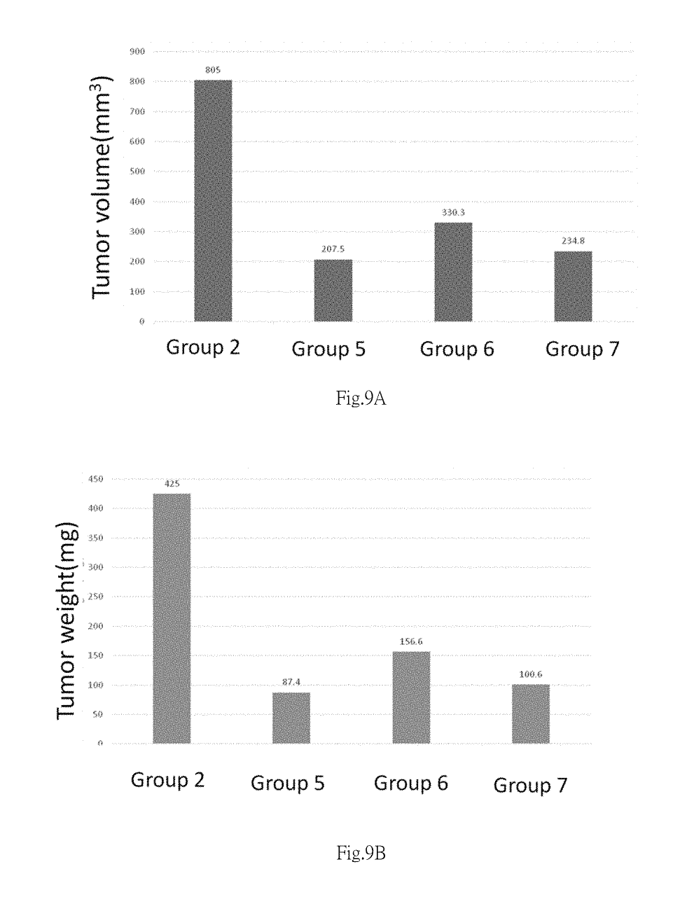

The test substance was administered during the experiment, and the tumor volume of the mouse was measured once or twice a week, and the longest (length) of the tumor and the widest (width) position perpendicular thereto were measured with a vernier scale. The formula is: length×width 2/2. The testing results were shown as FIG. 7, FIG. 8 and FIG. 9.

### 2. Survival Rate:

The test substance was given during the experiment, and the survival rate of the mice was observed 1 to 2 times per week.

### 3. Weight Change:

The test substance was administered during the experiment, and the mice were weighed 1 to 2 times a week, and measured by a weight machine and recorded.

### 4. End-Point Serum Cytokine Phenotyping:

At the end of the experiment, the blood of the mice was placed in a 1.5 ml centrifuge tube and allowed to stand at room temperature for 30 minutes. The first centrifugation was 4500 rpm, 10 min, 4° C., the second centrifugation 14000 rpm, 10 min, 4° C., and the serum was stored at −20° C. Cytokines in mouse serum were analyzed using a Milliplex MCYTOMAG-70K Kit (Millipore). For the analysis method, refer to the operation manual of Milliplex MAP kit (Millipore); after the serum sample is diluted according to the operation manual, 25 ul of the sample to be tested and the magnetic beads and standard products containing specific antibodies are separately added to the well plate, and placed in 4° C. refrigerator, raise overnight. The next day, the buffer solution was added for washing, and then the biotin label secondary antibody and PE-conjugated streptavidin were sequentially added, and protected from light, shaking, and culture at room temperature, and then the MAGPIX instrument was used for the analysis and data analysis. The IL-8 was used to evaluate the efficiency of the pharmaceutical composition of the present invention. In addition to being a chemokines, IL-8 also has many functions that promote tumor progression: it can be used as a growth factor for certain cancer cells, it can promote tumor angiogenesis, and it can even enhance the metastasis of cancer cells ability. Many literatures also support the association between IL-8 and nasopharyngeal carcinoma. Cytokines can be released by different cells and deliver different cell messages that alter the function of the cells. Cytokines, such as G-CSF(granulocyte colony-stimulating factor) can induce the white blood cells growth; IL-8 (interleukin-8), IL-6 (interleukin-6) and TNF-α are involved in the pathogenesis of leukemia, the decrease of these cytokines can reduce the inflammation.

### Example 8. Tumor Volume and Tumor Weight of Mice in 9 Groups Treatment

The tumor volume and tumor weight of mice in 9 groups were measured in 21th day of drug treatment. The results were shown in FIG. 7 to FIG. 9.

FIG. 7A shows the tumor volume of mice in group 2, group 3 and group 4; FIG. 7B shows the tumor weight of mice in group 2, group 3 and group 4. As shown in FIG. 7A, the tumor volume of mice in group 2 is 805 mm³, the tumor volume of mice in group 3 is 194.4 mm³, and the tumor volume of mice in group 4 is 95.7 mm³. The pharmaceutical composition of the present invention would decrease the tumor volume. As shown in FIG. 7B, the tumor weight of mice in group 2 is 425 mg, the tumor weight of mice in group 3 is 57.1 mg, and the tumor weight of mice in group 4 is 19.7 mg. The pharmaceutical composition of the present invention would decrease the tumor weight.

FIG. 8A shows the tumor volume of mice in group 2, group 8 and group 9; FIG. 8B shows the tumor weight of mice in group 2, group 8 and group 9. As shown in FIG. 8A, the tumor volume of mice in group 2 is 805 mm³, the tumor volume of mice in group 8 is 170.5 mm³, and the tumor volume of mice in group 9 is 180.4 mm³. The pharmaceutical composition of the present invention would decrease the tumor volume. As shown in FIG. 8B, the tumor weight of mice in group 2 is 425 mg, the tumor weight of mice in group 8 is 43.1 mg, and the tumor weight of mice in group 9 is 48.7 mg. The pharmaceutical composition of the present invention would decrease the tumor weight.

FIG. 9A shows the tumor volume of mice in group 2, group 5, group 6 and group 7; FIG. 9B shows the tumor weight of mice in group 2, group 5, group 6 and group 7. As shown in FIG. 9A, the tumor volume of mice in group 2 is 805 mm³, the tumor volume of mice in group 5 is 207.5 mm³, the tumor volume of mice in group 6 is 330.3 mm³ and the tumor volume of mice in group 7 is 234.8 mm³. The pharmaceutical composition of the present invention would decrease the tumor volume. As shown in FIG. 9B, the tumor weight of mice in group 2 is 425 mg, the tumor weight of mice in group 5 is 87.4 mg, the tumor weight of mice in group 6 is 156.6 mg, and the tumor weight of mice in group 7 is 100.6 mg. The pharmaceutical composition of the present invention would decrease the tumor weight.

### Example 9. The Expression of IL-8 in 9 Groups Treatment

The expression of IL-8 in 9 groups were measured in 21th day of drug treatment. The results were shown in FIG. 10 to FIG. 12.

FIG. 10 shows the expression of IL-8 in group 2, group 3 and group 4, which is 434, 77 and 4 (pg/ml) respectively. Compare to group 3, the expressions of cytokine IL-8 has effectively reduced in group 4.

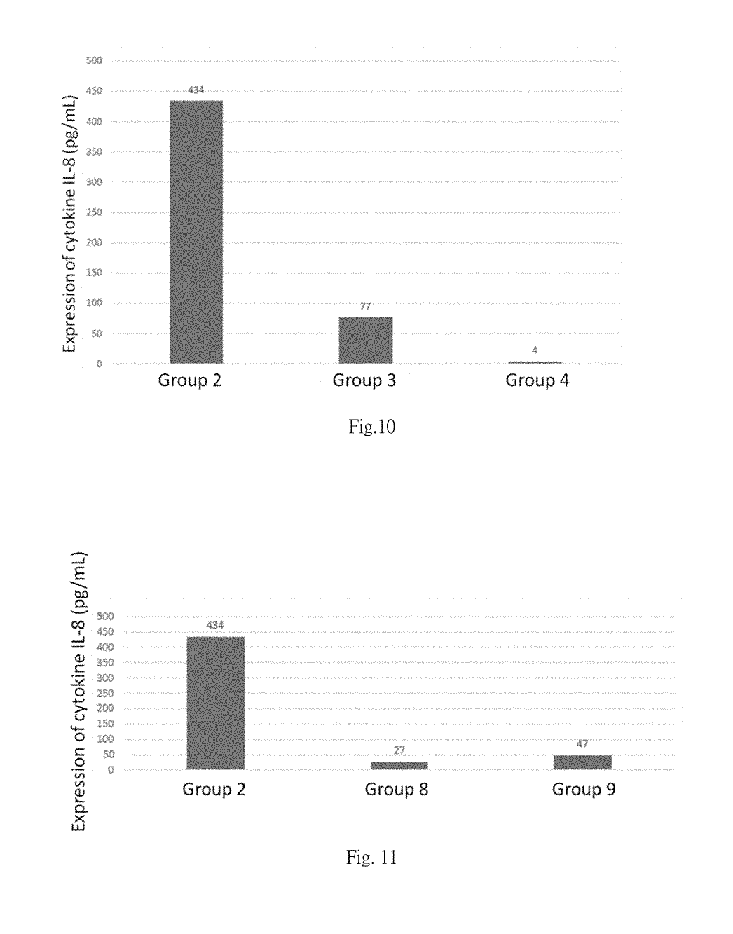

FIG. 11 shows the expression of IL-8 in group 2, group 8 and group 9, which is 434, 27 and 47 respectively (pg/ml). Compare to group 9, the expressions of cytokine IL-8 has effectively reduced in group 8.

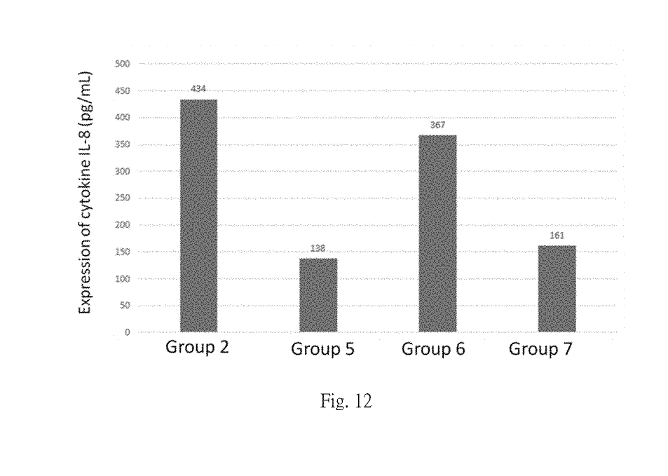

FIG. 12 shows the expression of IL-8 in group 2, group 5, group 6 and group 7, which is 434, 138, 367 and 161 (pg/ml)respectively. Compare to group 6 and group 7, the expressions of cytokine IL-8 has effectively reduced in group 5.

In summary, LV, 5FU, ONIVYDE, Tianxian Liquid, Chemo young and ZSAINO are all commonly used drugs in clinical practice, and the pharmaceutical composition of the present invention has better therapeutic effect. Moreover, Tianxian Liquid, Chemo young and ZSAINO are also Chinese herbal medicines, but the pharmaceutical composition of the present invention has better therapeutic effect. The pharmaceutical composition of the present invention have better effect on treating tumor than ONIDYVE, Chemo Young, ZSAINO.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A pharmaceutical kit for treating pancreatic cancer or decreasing the side effects of pancreatic cancer drug, comprising: a pancreatic cancer drug combination and a pharmaceutical composition,
wherein the pancreatic cancer drug combination is selected from the group consisting of a topoisomerase I inhibitor, a topoisomerase II inhibitor, an antimetabolite, and combinations thereof; and
a pharmaceutical composition, wherein the pharmaceutical composition comprises a mushroom, a rhizome, a fruit, a leaf, a flower, algae, an energy-rich liquid, a salt-rich liquid, an assist agent and an anti-oxide agent; wherein the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof; wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof; wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof; wherein the leaves are selected from the group consisting of 8-12 grams of spinach, 8-12 grams of sprouted broccoli, 8-12 grams of papaya leaves, 6.4-9.6 g of Lotus leaf and combinations thereof; wherein the flower system is 8-12 g of Gongju chrysanthemum, 8-12 g of Huang Ju, 8-12 g of honeysuckle, 8-12 of chamomile G, or a combination thereof; wherein the algae is a group consisting of 8-12 grams of seaweed, 8-12 grams of konbu and 8-12 grams of kelp, or a combination thereof; wherein the energy configuration fluid comprises chlorine 4.5-5.5 mg of iron and 200 ml of distilled water; wherein, the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride; wherein the supplement 18-22 grams of citric acid, 18-22 grams of selenium yeast, 270-330 milligrams of coenzyme, and 2.7-3.3 grams of vitamin C; and the antioxidant comprises 9-11 grams of chitosan oligosaccharides, −5.5 grams, 0.9-1.1 grams of pine bark and 0.9-1.1 grams of brown algae polysaccharide.

2. The pharmaceutical kit of claim 1, wherein the pharmaceutical composition has a pH between 1.2-2.

3. The pharmaceutical kit of claim 1, wherein the pancreatic cancer drug combination further comprising Leucovorin.

4. The pharmaceutical kit of claim 1, wherein the topoisomerase I inhibitor is irinotecan, SN-38 or topotecan;
wherein the topoisomerase II inhibitor is anthracycline, teniposide or etoposide; and
wherein the antimetabolite is 5-Fluorouracil, capecitabine, Cytarabine, gemcitabine, methotrexate, pemetrexed or tegafur.

5. A method for treating pancreatic cancer or decreasing the side effects of pancreatic cancer drug in a subject, wherein the method comprising administering to said subject an effective amount of a pharmaceutical composition, wherein the pharmaceutical composition comprises a mushroom, a rhizome, a fruit, a leaf, a flower, algae, an energy-rich liquid, a salt-rich liquid, an assist agent and an anti-oxide agent;
wherein the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof;
wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof;
wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof;
wherein the leaves are selected from the group consisting of 8-12 grams of spinach, 8-12 grams of sprouted broccoli, 8-12 grams of papaya leaves, 6.4-9.6 g of Lotus leaf and combinations thereof;
wherein the flower system is 8-12 g of Gongju chrysanthemum, 8-12 g of Huang Ju, 8-12 g of honeysuckle, 8-12 of chamomile G, or a combination thereof;
wherein the algae is a group consisting of 8-12 grams of seaweed, 8-12 grams of konbu and 8-12 grams of kelp, or a combination thereof;
wherein the energy configuration fluid comprises chlorine 4.5-5.5 mg of iron and 200 ml of distilled water;
wherein, the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride;
wherein the supplement 18-22 grams of citric acid, 18-22 grams of selenium yeast, 270-330 milligrams of coenzyme, and 2.7-3.3 grams of vitamin C; and
wherein the antioxidant comprises 9-11 grams of chitosan oligosaccharides, −5.5 grams, 0.9-1.1 grams of pine bark and 0.9-1.1 grams of brown algae polysaccharide.

6. The method of claim 5, wherein the administration of the pharmaceutical composition divided into 3 to 10 times a day and each time must be diluted 1/1000 to 3/1000 times.

7. The method of claim 5, wherein the pharmaceutical composition is used in combination with a course of a pancreatic cancer drug.

8. The method of claim 7, wherein the administration of the pharmaceutical composition is administered daily for several days prior to the course of the pancreatic cancer drug, then the course of the pancreatic cancer drug begins on the day when the pharmaceutical composition is discontinued, and then continue to daily administration of the pharmaceutical composition after the course of the pancreatic cancer drug.

9. The method of claim 7, wherein the administration of the pharmaceutical composition is administered daily for several days prior to the course of the pancreatic cancer drug, then continue daily administration of the pharmaceutical composition during the course of the pancreatic cancer drug, and then continue to daily administration of the pharmaceutical composition after the course of the pancreatic cancer drug.

10. The method of claim 7, wherein the administration of the pharmaceutical composition is administered daily for several days prior to the course of the pancreatic cancer drug, then continue daily administration of the pharmaceutical composition during the course of the pancreatic cancer drug, and then continue to daily administration of the pharmaceutical composition after the course of the pancreatic cancer drug, sequential administration as above.

11. The method of claim 7, wherein the pancreatic cancer drug is a antimetabolite or the antimetabolite and Leucovorin combination.

12. The pharmaceutical composition of claim 11, wherein the antimetabolite is 5-Fluorouracil, capecitabine, Cytarabine, gemcitabine, methotrexate, pemetrexed or tegafur.

13. A pharmaceutical composition for treating pancreatic cancer and decreasing the side effects of pancreatic cancer drug in a subject, wherein the pharmaceutical composition comprises a mushroom, a rhizome, a fruit, a leaf, a flower, algae, an energy-rich liquid, a salt-rich liquid, an assist agent and an anti-oxide agent;
wherein the mushroom is selected from the group consisting of 24-36 grams of Phellinus linteus, 16-24 grams of Ganoderma lucidum, 16-24 grams of Brazilian mushroom, 4-6 grams of Antrodia camphorata, 4-6 grams of Cordyceps sinensis and combinations thereof;
wherein the rhizome is selected from the group consisting of 16-24 grams of yellow Fine, 8-12 grams of Astragalus, 8-12 grams of Salvia, 8-12 grams of Codonopsis, 12-18 grams of Hedyotis diffusa, 12-18 grams of Eucommia, 8-12 grams of Atractylodes, 8-12 grams of TCS, 8-12 grams of Acanthopanax, 8-12 grams of Ophiopogon japonicus, 8-12 grams of Rhodiola rose, 2.4-3.6 grams of Pear licorice and combinations thereof;
wherein the fruit seeds 12-18 grams of barley, 8-12 grams of Fructus Schisandrae, 8-12 grams of Schisandra, 9.6-14.4 grams of germinated rice, 8-12 grams of lotus seed, 8-12 grams of black sesame, 8-12 grams of corn, 8-20 grams of Luo Han Guo −12 grams, 1.6-2.4 grams of red grape skin extract and combinations thereof;
wherein the leaves are selected from the group consisting of 8-12 grams of spinach, 8-12 grams of sprouted broccoli, 8-12 grams of papaya leaves, 6.4-9.6 g of Lotus leaf and combinations thereof; wherein the flower system is 8-12 g of Gongju chrysanthemum, 8-12 g of Huang Ju, 8-12 g of honeysuckle, 8-12 of chamomile G, or a combination thereof;
wherein the algae is a group consisting of 8-12 grams of seaweed, 8-12 grams of konbu and 8-12 grams of kelp, or a combination thereof; wherein the energy configuration fluid comprises chlorine 4.5-5.5 mg of iron and 200 ml of distilled water;
wherein the salt solution comprises 112.5-137.5 grams of deep sea salt, 27-33 grams of magnesium chloride, 18-22 milliliters of brine, 0.99-1.21 grams of calcium chloride, and 0.495-0.605 grams of potassium chloride; wherein the supplement 18-22 grams of citric acid, 18-22 grams of selenium yeast, 270-330 milligrams of coenzyme, and 2.7-3.3 grams of vitamin C; and
wherein the antioxidant comprises 9-11 grams of chitosan oligosaccharides, −5.5 grams, 0.9-1.1 grams of pine bark and 0.9-1.1 grams of brown algae polysaccharide.

14. The method of claim 13, wherein the administration of the pharmaceutical compositions divided into 3 to 10 times a day and each time must be diluted 1/1000 to 3/1000 times.

15. The method of claim 13, wherein the pharmaceutical compositions is used in combination with a course of a pancreatic cancer drug.

16. The method of claim 15, wherein the administration of the pharmaceutical composition is administered daily for several days prior to the course of the pancreatic cancer drug, then the course of the pancreatic cancer drug begins on the day when the pharmaceutical composition is discontinued, and then continue to daily administration of the pharmaceutical composition after the course of the pancreatic cancer drug.

17. The method of claim 15, wherein the administration of the pharmaceutical composition is administered daily for several days prior to the course of the pancreatic cancer drug, then continue daily administration of the pharmaceutical composition during the course of the pancreatic cancer drug, and then continue to daily administration of the pharmaceutical composition after the course of the pancreatic cancer drug.

18. The method of claim 15, wherein the administration of the pharmaceutical composition is administered daily for several days prior to the course of the pancreatic cancer drug, then continue daily administration of the pharmaceutical composition during the course of the pancreatic cancer drug, and then continue to daily administration of the pharmaceutical composition after the course of the pancreatic cancer drug, sequential administration as above.

19. The method of claim 15, wherein the pancreatic cancer drug is a antimetabolite or the antimetabolite and Leucovorin combination.

20. The method of claim 16, wherein the antimetabolite is 5-Fluorouracil, capecitabine, Cytarabine, gemcitabine, methotrexate, pemetrexed or tegafur.
